(19) United States
(12) Patent Application Publication
(10) Pub. No.: US 2011/0217373 A1
(43) Pub. Date: Sep. 8, 2011

(54) EXTENDED RELEASE PHARMACEUTICAL COMPOSITIONS OF GUANFACINE HYDROCHLORIDE

(76) Inventors: Kumaravel VIVEK, Chennai (IN); Anuj Kumar Fanda, Ghaziabad (IN); Sweta Varshney, Ghaziabad (IN); Romi Barat Singh, Varanasi (IN); Lalit Khurana, Chandigarh (IN); Narravula Sreekanth, Guntur (IN)

(21) Appl. No.: 13/017,579

(22) Filed: Jan. 31, 2011

(30) Foreign Application Priority Data
Jan. 29, 2010 (IN) 207/DEL/2010

(51) Int. Cl. A61K 9/24; A61K 31/165; A61P 25/00

(52) U.S. Cl. 424/465; 514/617

(57) ABSTRACT

The present invention relates to an extended release pharmaceutical tablet composition comprising guanfacine comprising: a core containing guanfacine or a pharmaceutically acceptable salt thereof and one or more of pH-independent rate controlling polymer(s) and other pharmaceutically acceptable excipients; optionally a coating over the core as in (a) wherein, the coating comprises one or more of pH-independent rate controlling polymer(s).

### TECHNICAL FIELD OF INVENTION

The present invention relates to extended release pharmaceutical tablet composition of guanfacine hydrochloride and processes for the preparation of the same.

### BACKGROUND OF THE INVENTION

Guanfacine hydrochloride is a selective α_{2A}-adrenergic agonist and is useful for the treatment of attention deficit hyperactivity disorder (ADHD). The chemical name of guanfacine hydrochloride is N-amidino-2-(2,6-dichlorophenyl) acetamide monohydrochloride. U.S. Pat. No. 3,632,645 discloses guanfacine and it's pharmaceutically acceptable acid addition salts specifically. The method of treating a behavioral disinhibition (e.g. Attention-Deficit Hyperactivity Disorder) in a primate with minimal sedative side effects by administering thereto a therapeutically effective amount of guanfacine is disclosed in U.S. Pat. No. 5,854,290.

Guanfacine is marketed under the brand name Intuniv® from Shire Pharmaceuticals and is available in four strengths: 1 mg, 2 mg, 3 mg and 4 mg extended release tablets. These tablets contain guanfacine in a matrix that includes hydroxypropylmethyl cellulose, methacrylic acid copolymer, lactose, povidone, crospovidone, microcrystalline cellulose, fumaric acid, and glyceryl behenate as described in U.S. Pat. Nos. 6,287,599 and 6,811,794; both of which are assigned to Shire Laboratories Inc., and are incorporated herein by reference. The extended release pharmaceutical composition as described by these patents require at least one pH dependent agent that increases the rate of release of guanfacine hydrochloride from the tablet dosage form at a pH in excess of 5.5 to maintain a pH-independent or a minimized pH-dependent release profile. Such pH dependent agents include enteric agents, polymers that swell above pH 5.5 and agents that increase the solubility of guanfacine hydrochloride above pH 5.5, like certain organic acids.

The inventors of the present invention have discovered that it is possible to prepare extended release pharmaceutical compositions without the use of any pH dependent agent that increases the rate of release of guanfacine hydrochloride from the composition at a pH in excess of 5.5 and yet achieve a pH-independent or a minimized pH dependent dissolution profile.

WO 2007/016284 discloses methods and compositions that are useful in the treatment of any of the indications for guanfacine. The methods include administering to a subject a once a day, oral therapeutic composition or formulation containing guanfacine in the prescribed dose, e.g., 1 mg, 1.5 mg, 2 mg, 2.5 mg, 3 mg, 3.5 mg, etc., in a single dose form, e.g., a single tablet, which is effective in a once a day regimen and also has a size small enough to be acceptable for oral administration. This application also discloses a pharmaceutical composition comprising guanfacine and a pharmaceutically acceptable vehicle in a single, once a day discrete dosage form for oral administration. For example, typical acceptable sizes, in terms of tablet weight, are for a 1 mg dose, up to 170 mg, for a 2 mg dose, up to 340 mg, for a 2.5 mg dose, up to 255 mg, for a 3 mg dose, up to 225 mg, for a 3.5 mg dose, up to 245 mg, and for a 4 mg dose, up to 300 mg.

WO 2007/016350 discloses a method of formulating guanfacine hydrochloride in a solid dosage form of a specified hardness, which comprises guanfacine hydrochloride and at least one non-pH dependent sustained release agent, the method comprising selecting an amount of Eudragit® L100-55 specifically designed to achieve said specified hardness.

WO 2004/062577 discloses a pharmaceutical composition comprising a core containing guanfacine, and a coating layer surrounding the core, said coating layer comprising a combination of two or more enteric coating materials, at least two of which enteric coating materials will dissolve at different pH's to give pH-dependent release profile.

### SUMMARY OF THE INVENTION

In one general aspect, the present invention provides for an extended release pharmaceutical tablet composition of guanfacine, which includes:
a) a core containing guanfacine or a pharmaceutically acceptable salt thereof and one or more of pH-independent rate controlling polymer(s) and other pharmaceutically acceptable excipients; and
b) optionally, a coating over the core of (a) wherein, the coating comprises one or more of pH-independent rate controlling polymer(s).

In another general aspect, the present invention provides for an extended release pharmaceutical tablet composition of guanfacine, which includes:
a) an inert pellet;
b) a layer surrounding the inert pellet of (a), wherein the layer comprises guanfacine or a pharmaceutically acceptable salt thereof and other pharmaceutically acceptable excipients; and
c) a coating surrounding the layer of (b), wherein the coating comprises one or more of pH-independent rate controlling polymer(s) and other pharmaceutically acceptable excipients.

In yet another general aspect, the present invention provides for an extended release pharmaceutical tablet composition of guanfacine, which includes guanfacine or a pharmaceutically acceptable salt thereof, one or more of sustained release wax, one or more of pH-independent rate controlling polymer(s) and other pharmaceutically acceptable excipients.

Embodiments of the present invention may include one or more of the following features. For example, the core may further include an acidic microenvironment pH modifier and/or enteric agent(s). The core may further include a gastro-soluble cationic polymethacrylate copolymer. For example, the core further include carboxyvinyl polymer.

Suitable pH-independent rate controlling polymer(s) include one or more of guar gum, acacia gum, tragacanth gum, xanthum gum, hydroxyethyl cellulose, hydroxypropyl cellulose, hydroxypropyl methylcellulose, carboxymethyl cellulose, sodium carboxymethyl cellulose, carbopol, polycarbofil, vinyl acetate copolymers, methacrylic acid copolymers, maleic anhydride-methyl vinyl ether copolymers, acrylates, ethylcellulose, methacrylates, acrylic acid copolymers, high molecular weight polyvinyl alcohols, stearyl alcohol, glyceryl palmitostearate, gleceryl monostearate, carnuba wax, beeswax, candelilla wax, microcrystalline wax, ozokerite wax, paraffin wax, glyceryl behenate and hydrogenated vegetable oil.

Suitable acidic microenvironment pH modifiers includes one or more of hydrochloric acid, phosphoric acid, nitric acid, sulphuric acid, fumaric acid, citric acid, L-cysteine hydrochloride, glycine hydrochloride, ascorbic acid, butylated hydroxyanisol, butylated hydroxytoluene, salts of organic bases, salts of inorganic acids, salts of organic acids and acidic buffers.

Suitable enteric agent(s) include one or more of hydroxypropyl methylcellulose phthalate, cellulose acetate phthalate, and polyvinyl acetate phthalate.

Suitable other pharmaceutically acceptable excipients include one or more of binder(s), disintegrant(s), lubricant(s), diluent(s), glidants(s), surfactant(s), and solvent(s).

### DETAILED DESCRIPTION OF THE INVENTION

The term “extended release pharmaceutical composition”, as referred to herein, is defined as pharmaceutical compositions for oral administration which provide plasma concentrations of guanfacine or a pharmaceutically acceptable salt thereof that remains substantially invariant over time within the therapeutic range over a 24-hour period thereby mitigating the side effects. This definition encompasses “controlled release”, “modified release”, “prolonged release”, “delayed release” and “sustained release” compositions. The compositions according to the present invention deliver a therapeutically effective amount of guanfacine or a pharmaceutically acceptable salt thereof to a patient for 24 hours following a once-daily administration.

The term “therapeutically effective amount” intends to describe an amount of the guanfacine or a pharmaceutically acceptable salt thereof which reduces, eliminates, treats, prevents or controls the symptoms of the disease conditions to be treated in a human patient. Guanfacine or a pharmaceutically acceptable salt thereof may be present in an amount from about 0.1% to about 70% by weight of the extended release pharmaceutical composition. For example, it may be present in an amount from about 0.5% to about 40% by weight of the extended release pharmaceutical composition. The recommended dose of Intuniv® may be considered as a standard dose.

The term “pharmaceutically acceptable salt” as used herein refers to inorganic acid salts such as hydrochloride, hydrobromide, sulfate and phosphate; organic acid salts, such as, acetate, maleate, fumarate, tartrate and citrate. In one of the embodiments, the salt form of guanfacine may be guanfacine hydrochloride.

The “pH-independent rate controlling polymers” may include hydrophilic and/or hydrophobic polymers. The hydrophilic polymers may include carbohydrate gum selected from one or more of guar gum, acacia gum, tragacanth gum, xanthan gum; cellulose ether selected from one or more of hydroxyethyl cellulose, hydroxypropyl cellulose, hydroxypropyl methyl cellulose, carboxymethyl cellulose, sodium carboxy methyl cellulose; acrylic acid polymer selected from one or more of carboxy vinyl polymer, carbopol, polycarbofil; vinyl acetate copolymers; methacrylic acid copolymers; maleic anhydride/methyl vinyl ether copolymers and other hydrophilic polymers known to those skilled in the art or a derivative or a mixture thereof. The hydrophobic polymers may include acrylates, cellulose derivatives such as ethylcellulose or cellulose acetate, methacrylates, acrylic acid copolymers, high molecular weight polyvinylalcohols, stearyl alcohol, glyceryl palmitostearte, glyceryl monostearate; and waxes selected from one or more of carnauba wax, beeswax, candelilla wax, microcrystalline wax, ozokerite wax, paraffin waxes glyceryl behenate, hydrogenated vegetable oil and other hydrophobic polymers known to those skilled in the art or a derivative or a mixture thereof. The pH-independent rate controlling polymers may be present in an amount ranging from about 0.1% to about 90% by weight of the extended release pharmaceutical composition. For example, it may be present in an amount ranging from about 0.5% to about 70% by weight of the extended release pharmaceutical composition.

The term “sustained release wax” may include plant or animal wax selected from carnauba wax, bees wax; various hydrogenated oils selected from hydrogenated soybean oil, hydrogenated castor oil; paraffin's selected from paraffin wax, microcrystalline wax and the like. The wax, as mentioned above, may be used as a mixture thereof. The sustained release wax may be present in an amount ranging from about 1% to about 90% by weight of the extended release pharmaceutical composition. For example, it may be present in an amount ranging from about 5% to about 70% by weight of the extended release pharmaceutical composition.

The term “acidic microenvironment pH modifier”, as referred to herein, is defined as acidic agents that modify the microenvironment of the extended release pharmaceutical composition without increasing the rate of release of guanfacine or a pharmaceutically acceptable salt thereof at a pH in excess of 5.5. U.S. Pat. Nos. 6,811,794 and 6,287,599 disclose pH dependent agents that increase the rate of release of guanfacine hydrochloride at a pH in excess of 5.5 by maintaining an acidic microenvironment in the tablet; however, the acidic microenvironment pH modifier, referred to as herein, does not increase the rate of release of guanfacine hydrochloride at a pH in excess of 5.5. Suitable pH modifiers may include inorganic acids selected from one or more of hydrochloric acid, phosphoric acid, nitric acid and sulphuric acid; organic acids selected from one or more of fumaric acid and citric acid; salts of amino acids selected from one or more of L-cysteine hydrochloride and glycine hydrochloride; antioxidants selected from one or more of ascorbic acids, butylated hydroxyanisole and butylated hydroxytoluene; salts of organic bases; salts of inorganic acids; salts of organic acids; and acidic buffers. The acidic microenvironment pH modifiers may be present in an amount ranging from about 0.01% to about 25% by weight of the extended release pharmaceutical composition. For example, it may be present in an amount ranging from about 0.05% to about 15% by weight of the extended release pharmaceutical composition.

The term “enteric agents”, as referred to herein, may further help in providing the desired dissolution profile from the extended release pharmaceutical composition without increasing the rate of release of guanfacine or a pharmaceutically acceptable salt thereof at a pH in excess of 5.5. The enteric agents include hydroxypropyl methylcellulose phthalate, cellulose acetate phthalate, polyvinyl acetate phthalate. The enteric agents may be present in an amount ranging from about 1% to about 50% by weight of the extended release pharmaceutical composition. For example, it may be present in an amount ranging from about 5% to about 40% by weight of the extended release pharmaceutical composition.

The term “gastro-soluble cationic polymethacrylate copolymer”, as referred to herein, includes Eudragit® EPO.

The term “carboxyvinyl polymer”, as referred to herein, are polymers which are insoluble but swellable in water and may further assist to attain the desired dissolution profile from the extended release pharmaceutical composition without increasing the rate of release of guanfacine or a pharmaceutically acceptable salt thereof at a pH in excess of 5.5. The carboxyvinyl polymer includes carbopol.

The term “pharmaceutically acceptable excipients”, as referred to herein, includes conventional pharmaceutical additives known in the art, such as binder(s), disintegrant(s), lubricant(s), diluent(s), glidant(s), surfactant(s), solvent(s) or combinations thereof.

Binders that may be used include starch derivatives like corn starch and pregelatinized starch; cellulose ethers such as carboxymethyl cellulose, methylcellulose, hydroxypropyl cellulose, ethylcellulose, hydroxypropyl methylcellulose; carboxy vinyl polymers like carbomers; acrylates, such as eudragits; polyvinylpyrrolidone; polyvinylpyrrolidone/vinyl acetate copolymer; xanthan gum, guar gum. The binder may be present in an amount ranging from about 0.1% to about 25% by weight of the extended release pharmaceutical composition. For example, in an amount ranging from about 0.5% to about 15% by weight of the extended release pharmaceutical composition. In one embodiment, the binder is polyvinylpyrrolidone.

Disintegrants that may be used include croscarmellose sodium, sodium starch glycolate, microcrystalline cellulose, crospovidone, polyvinyl pyrrolidone, low-substituted hydroxypropylcellulose, alginic acid, calcium salts and potassium salts of carboxymethyl cellulose, colloidal silicon dioxide, guar gum, magnesium aluminum silicate, methylcellulose, powdered cellulose, starch, and sodium alginate. The disintegrant may be present in an amount ranging from about 1% to about 30% by weight of the extended release pharmaceutical composition. For example, in an amount ranging from about 5% to about 25% by weight of the extended release pharmaceutical composition.

Lubricants that may be used include magnesium stearate, calcium stearate, zinc stearate, sodium stearyl fumarate, powdered stearic acid, magnesium oleate, calcium palmitate, potassium laureate, sodium suberate, vegetable oil, mineral oil, talc, beeswax, carnuba wax, glyceryl stearate, glyceryl palmitate, glyceryl behenate, hydrogenated vegetable oils and the like. The lubricant may be present in an amount ranging from about 0.1% to about 20% by weight of the extended release pharmaceutical composition. For example, in an amount ranging from about 0.5% to about 10% by weight of the extended release pharmaceutical composition.

Diluents that may be used include saccharides like lactose, dextrose, sucrose, fructose, maltose; sugars like mannitol, erythritol, sorbitol, xylitol and lactitol; cellulose derivatives like powdered cellulose, microcrystalline cellulose; dicalcium phosphate, tribasic calcium phosphate, calcium sulphate, calcium carbonate, kaolin and the like. The diluent may be present in an amount ranging from about 5% to about 90% by weight of the extended release pharmaceutical composition; for example, in an amount ranging from about 5% to about 60% by weight of the extended release pharmaceutical composition.

Glidants that may be used include colloidal silicon dioxide, corn starch, and the like. The glidant may be present in an amount ranging from about 0.2% to about 20% by weight of the extended release pharmaceutical composition; for example, in an amount ranging from about 0.5% to about 10% by weight of the extended release pharmaceutical composition.

Surfactants that may be used include sodium lauryl sulphate.

Suitable solvents that may be used include water, ethanol, methanol, isopropyl alcohol, methylene chloride, acetone, and the like.

The extended release pharmaceutical compositions may be administered orally in a once daily regimen in the form of tablets, capsules, pellets, beads, pills or granules. The tablets may be prepared by techniques that are well known in the art including direct compression, dry granulation, or wet granulation.

The extended release pharmaceutical compositions manufactured using direct compression requires a process by which powder blend of an active ingredient, and a suitable excipient and/or filler, which is capable of flowing uniformly, are compressed directly into an acceptable tablet.

The extended release pharmaceutical compositions manufactured using dry granulation requires two compaction steps. The first occurs during roller compaction or slugging, when the granulation binder-containing formulation is compacted to form granules. The second occurs during formation of the solid dosage form, or tableting, when the tablet formulation, which contains the granules, is compacted into a tablet.

The extended release pharmaceutical compositions manufactured using wet granulation requires a process of using a liquid binder to lightly agglomerate the powder mixture to form granules. These granules may be mixed with other suitable excipients and compacted into a acceptable tablet.

The extended release pharmaceutical tablet compositions may be additionally prepared with a coating with one of the commercially available coating systems or any one of polymeric film coatings used in the formulation of pharmaceutical compositions. The coating generally includes film forming polymers such as ethyl cellulose, hydroxypropyl methylcellulose, hydroxypropyl cellulose, methylcellulose, carboxymethyl cellulose, hydroxyl methylcellulose, cellulose acetate, methacrylic acid polymers and combinations thereof; fillers such as talc, lactose; plasticizers such as polyethylene glycol, and the like; and antioxidants such as butylated hydroxyl toluene and butylated hydroxyl anisole, lubricants like magnesium stearate and glidants like colloidal silicon dioxide.

In one embodiment, an extended release pharmaceutical tablet composition comprising guanfacine is prepared by the following steps:
a) guanfacine or a pharmaceutically acceptable salt thereof is mixed with one or more of diluent(s) and binder(s);
b) the mixture of step (a) is passed through a compactor to form drug-diluent compacts;
c) the drug-diluent compacts of step (b) are milled to form suitable size granules;
d) the granules of step (c) are mixed with one or more of pH-independent rate controlling polymer(s), optionally an acidic microenvironment pH modifier and one or more of lubricant(s) and glidant(s) to form the final blend; and
e) the final blend of step (d) is compressed into tablets using suitable tooling.

In another embodiment, an extended release pharmaceutical tablet composition comprising guanfacine is prepared by the following steps:
a) guanfacine or a pharmaceutically acceptable salt thereof is granulated with one or more of binder(s);
b) the granules of step (a) is mixed with one or more of pH-independent rate controlling polymer(s), optionally an acidic microenvironment pH modifier and one or more of diluent(s), disintegrant(s), lubricant(s), glidant(s) to form the final blend; and
c) the final blend of step (b) is compressed into tablets using suitable tooling.

In another embodiment, an extended release pharmaceutical tablet composition comprising guanfacine is prepared by the following steps:
a) guanfacine or a pharmaceutically acceptable salt thereof is mixed with one or more of diluent(s) and optionally an acidic microenvironment pH modifier to form a blend;
b) the blend of step (a) is granulated with one or more of binder(s) to form granules;
c) the granules of step (b) is mixed with one or more of lubricant(s), glidant(s) to form the final blend;
d) the final blend of step (c) is compressed into tablets using suitable tooling; and
e) the tablet of step (d) is compression coated with a coating composition comprising one or more of pH-independent rate controlling polymer(s).

In another embodiment, an extended release pharmaceutical tablet composition comprising guanfacine is prepared by the following steps:

a)
i. guanfacine or a pharmaceutically acceptable salt thereof, optionally an acidic microenvironment pH modifier and one or more of binder(s) are coated onto inert pellets to form drug layered pellets; and
ii. the drug layered pellets of step (i) are further coated with one or more of pH-independent rate controlling polymer(s) to a weight gain level which gives fast release pellets.

b)
i. guanfacine or a pharmaceutically acceptable salt thereof, optionally an acidic microenvironment pH modifier and one or more of binder(s) are coated onto inert pellets to form drug layered pellets; and
ii. the drug layered pellets of step (i) are further coated with one or more of pH-independent rate controlling polymer(s) to a weight gain level which gives slow release pellets.

c)
i. the pellets from steps (a) and (b) are mixed with one or more of diluent(s), disintegrant(s), surfactant(s), lubricant(s) and glidant(s) to form the final blend; and
ii. the final blend of step (i) is compressed into tablets using suitable tooling.

In another embodiment, an extended release pharmaceutical tablet composition comprising guanfacine is prepared by the following steps:
a) guanfacine or a pharmaceutically acceptable salt thereof is dispersed into a hot melt comprising one or more of sustained release polymer(s) to form a drug-wax melt;
b) the drug-wax melt of step (a) is milled to form granules of suitable size;
c) the granules of step (b) is mixed with one or more of pH-independent rate controlling polymer(s), optionally an acidic microenvironment pH modifier and one or more of diluent(s), lubricant(s), and glidant(s) to form the final blend; and
d) the final blend of step (c) is compressed into tablets using suitable tooling.

In another embodiment, an extended release pharmaceutical tablet composition comprising guanfacine is prepared by the following steps:
a) guanfacine or a pharmaceutically acceptable salt thereof is mixed with one or more of diluent(s), disintegrant(s) and optionally an acidic microenvironment pH modifier and/or enteric agent(s) to form a blend;
b) the blend of step (a) is granulated with one or more of binder(s) to form granules;
c) the granules of step (b) is mixed with one or more of pH-independent rate controlling polymer(s), lubricant(s), glidant(s) and optionally an acidic microenvironment pH modifier and/or enteric agent(s) to form the final blend; and
d) the final blend of step (c) is compressed into tablets using suitable tooling.

In another embodiment, an extended release pharmaceutical tablet composition comprising guanfacine is prepared by the following steps:
a) guanfacine or a pharmaceutically acceptable salt thereof is mixed with one or more of diluent(s), disintegrant(s), one or more of pH-independent rate controlling polymer(s) and optionally an acidic microenvironment pH modifier and/or enteric agent(s) to form a blend;
b) the blend of step (a) is granulated with one or more of binder(s) to form granules;
c) the granules of step (b) is mixed with one or more of lubricant(s) and glidant(s) to form the final blend; and
d) the final blend of step (c) is compressed into tablets using suitable tooling.

In another embodiment, an extended release pharmaceutical tablet composition comprising guanfacine is prepared by the following steps:
a) guanfacine or a pharmaceutically acceptable salt thereof is mixed with one or more of diluent(s), disintegrant(s), binder(s), one or more of pH-independent rate controlling polymer(s) and carboxyvinyl polymer to form a blend;
b) the blend of step (a) is mixed with one or more of lubricant(s) and glidant(s) to form the final blend; and
c) the final blend of step (b) is compressed into tablets using suitable tooling.

In yet another embodiment, an extended release pharmaceutical tablet composition comprising guanfacine is prepared by the following steps:
a) guanfacine or a pharmaceutically acceptable salt thereof is mixed with one or more of diluent(s), disintegrant(s), one or more of pH-independent rate controlling polymer(s) and carboxyvinyl polymer to form a blend;
b) the blend of step (a) is granulated with one or more of binder(s) to form granules;
c) the granules of step (b) is mixed with one or more of lubricant(s) and glidant(s) to form the final blend; and
d) the final blend of step (c) is compressed into tablets using suitable tooling.

The following examples illustrates extended release guanfacine compositions and processes of making the compositions, however, the examples are merely provided to illustrate the compositions and processes for their preparation and are not intended to be limiting the invention.

### Examples 1 to 3

| | | Quantity (% w/w) | | |
|---|---|---|---|---|
| S.N. | Ingredients | Example 1 | Example 2 | Example 3 |
| 1. | Guanfacine HCl | 0.5-3 | 0.5-3 | 0.5-3 |
| 2. | Hydroxypropyl methylcellulose | 30-50 | 30-50 | 30-50 |
| 3. | Lactose monohydrate | 20-40 | 20-40 | 20-40 |
| 4. | Hydrochloric acid | — | — | 0.1-10 |
| 5. | Glycine HCl | 0.1-10 | 0.1-10 | |
| 6. | Polyvinyl pyrollidone | 3-8 | 3-8 | 3-8 |
| 7. | Magnesium stearate | 1-2 | 1-2 | 1-2 |
| 8. | Eudragit ® EPO | — | — | 1-10 |

Coating

| | | | | |
|---|---|---|---|---|
| 9. | Ethyl cellulose | 5-10 | — | 5-10 |
| 10. | Triethyl citrate | 0.5-3 | — | 0.5-3 |
| 11. | Polyvinyl pyrrolidone | 2-5 | — | 2-5 |
| 12. | Opadry ® | — | 5-10 | — |
| 13. | Isopropyl alcohol | QS | — | QS |
| 14. | Purified water | QS | QS | QS |

### Procedure:

The sustained release guanfacine hydrochloride compositions as per Examples 1 to 3 may be prepared by tableting procedures, such as direct compression, dry granulation, or wet granulation as follows:

### 1. Direct Compression:

Guanfacine hydrochloride is dry mixed with hydroxypropyl methylcellulose, Eudragit® EPO, lactose monohydrate, glycine HCl and polyvinyl pyrrolidone to form a blend. The blend thus formed is lubricated with magnesium stearate. The lubricated blend is compressed into tablets using a suitable tooling. The compressed tablets thus obtained are coated with coating materials as per the formulas given above.

### 2. Dry Granulation:

Guanfacine hydrochloride is dry mixed with lactose monohydrate and polyvinyl pyrrolidone to form a blend. The blend thus formed is passed through a compacter to obtain drug-diluent compacts. The drug-diluent compacts thus obtained are milled into granules of suitable size. The granules thus formed are mixed with hydroxypropyl methyl cellulose, Eudragit® EPO, glycine HCl and are further lubricated with magnesium stearate. The lubricated blend is compressed into tablets using a suitable tooling. The compressed tablets thus obtained are coated with coating materials as per the formulas given above.

### 3. Wet Granulation:

Guanfacine hydrochloride is dry mixed with hydroxypropyl methyl cellulose, Eudragit® EPO, hydrochloric acid and lactose monohydrate to form a blend. The blend thus obtained is granulated with polyvinyl pyrrolidone. The granules thus obtained are dried and suitably sized by milling. The dried granules are lubricated with magnesium stearate and compressed into tablets using suitable tooling. The compressed tablets thus obtained are coated with coating materials as per the formulas given above.

### Example 4

| S.N. | Ingredients | Quantity (% w/w) |
|---|---|---|
| 1. | Guanfacine HCl | 0.5-3 |
| 2. | Ethyl Cellulose | 30-50 |
| 3. | Hydroxypropyl Methyl Cllulose | 20-40 |
| 4. | Hydrochloric Acid | 0.1-10 |
| 5. | Lactose Monohydrate | 10-20 |
| 6. | Polyvinyl Pyrrolidone | 3-8 |
| 7. | Magnesium Stearate | 1-2 |

Coating

| | | |
|---|---|---|
| 8. | Opadry ® | 5-10 |
| 9. | Purified Water | QS |

### Procedure:

Guanfacine hydrochloride is granulated with ethyl cellulose. The granules thus obtained are mixed with hydroxypropyl methyl cellulose, hydrochloric acid, lactose monohydrate and polyvinyl pyrrolidone to form a powder blend. The powder blend thus obtained is lubricated with magnesium stearate and compressed into tablets using suitable tooling. The compressed tablets thus obtained are coated with coating materials as per the formula given above.

### Example 5

| S.N. | Ingredients | Quantity (% w/w) |
|---|---|---|
| 1. | Guanfacine HCl | 0.5-3 |
| 2. | Lactose Monohydrate | 20-40 |
| 3. | Polyvinyl Pyrrolidone | 3-8 |
| 4. | Magnesium Stearate | 1-2 |
| 5. | Hydrochloric Acid | 0.1-10 |

Compression coating

| | | |
|---|---|---|
| 6. | Ethyl Cellulose | 10-20 |
| 7. | Polyethylene Glycol | 5-10 |
| 8. | Butylated Hydroxyltoluene | 0.01-0.02 |
| 9. | Magnesium Stearate | 1-2 |
| 10. | Colloidal Silicon Dioxide | 1-2 |

### Procedure:

Guanfacine hydrochloride is granulated with lactose monohydrate using dilute hydrochloric acid to form granules. The granules thus obtained are further granulated with polyvinyl pyrrolidone to form final granules. The final granules thus obtained are lubricated with magnesium stearate and compressed into tablets using suitable tooling. The compressed tablets thus obtained are compression coated with coating composition obtained by mixing ethylcellulsoe, polyethylene glycol, butylated hydroxyl toluene, magnesium stearate and colloidal silicon dioxide.

### Example 6

| | | |
|---|---|---|
| S.N. | Ingredients | Quantity (% w/w) |

Fast release pellets

| | | |
|---|---|---|
| 1. | Sugar Spheres | 5-10 |
| 2. | HPMC | 0.5-2 |
| 3. | Guanfacine HCl | 0.5-1 |
| 4. | Sodium Lauryl Sulphate | 0.25-0.5 |
| 5. | Ethyl Cellulose | 0.5-10 |
| 6. | HPMC | 0.5-2 |
| 7. | Talc | 5-10 |
| 8. | Polyethylene Glycol | 5-10 |

Slow release pellets

| | | |
|---|---|---|
| 1. | Sugar Spheres | 5-10 |
| 2. | HPMC | 0.5-2 |
| 3. | Guanfacine HCl | 0.5-1 |
| 4. | Sodium Lauryl Sulphate | 0.25-0.5 |
| 5. | Ethyl Cellulose | 0.5-10 |
| 6. | HPMC | 0.5-2 |
| 7. | Talc | 5-10 |
| 8. | Polyethylene Glycol | 5-10 |

Tablet excipients

| | | |
|---|---|---|
| 1. | Lactose Monohydrate | 20-30 |
| 2. | Croscarmellose Sodium | 10-20 |
| 3. | Colloidal Silicon Dioxide | 1-3 |

Coating

| | | |
|---|---|---|
| 1. | Opadry ® | 5-10 |
| 2. | Purified Water | Q.S |

### Procedure:

A. Guanfacine hydrochloride, hydroxypropyl methylcellulose, talc, and polyethylene glycol are mixed together and are coated on sugar spheres to form drug loaded pellets.
B. One set of drug loaded pellets of step A are further coated with ethyl cellulose to lower weight gain levels to obtain fast release pellets.
C. Another set of drug loaded pellets of step A are further coated with ethyl cellulose to higher weight gain levels to obtain slow release pellets.
D. The pellets of steps B and C are mixed with polyvinylactose monohydrate, croscarmellose sodium and colloidal silicon dioxide and compressed into tablets using suitable tooling. The compressed tablets thus obtained are coated with coating materials as per the formula given above.

### Example 7

| S.N. | Ingredients | Quantity (% w/w) |
|---|---|---|
| 1. | Guanfacine HCl | 0.5-1 |
| 2. | Lactose Monohydrate | 5-50 |
| 3. | Hydrogenated Castor oil | 10-50 |
| 4. | HPMC | 1-50 |
| 5. | Hydrochloric Acid | 1-10 |
| 6. | Colloidal Silicon Dioxide | 0.1-2 |
| 7. | Talc | 0.1-2 |
| 8. | Magnesium Stearate | 0.5-2 |

Coating

| | | |
|---|---|---|
| 1. | Opadry ® | 1-4 |
| 2. | Purified Water | QS |

### Procedure:

Guanfacine hydrochloride is dispersed in a hot melt of hydrogenated castor oil to form a drug-wax melt. The drug-wax melt thus obtained is cooled and suitably sized by milling to obtain granules. The granules are mixed with lactose monohydrate, hydroxypropyl methyl cellulose and hydrochloric acid to obtain a blend. The blend is lubricated with magnesium stearate, talc and colloidal silicon dioxide and compressed into tablets using suitable tooling. The compressed tablets thus obtained are coated with coating materials as per the formula given above.

### Example 8

| S. | | A | B | C* |
|---|---|---|---|---|
| N. | Ingredients | % w/w | % w/w | % w/w |
| 1. | Guanfacine HCl | 1.73 | 1.83 | 2.48 |
| 2. | Lactose | 19.80 | 20.85 | 28.37 |
| 3. | Polyvinyl Pyrrolidone | 1.51 | 1.59 | 2.17 |
| 4. | Microcrystalline Cellulose | 21.55 | 22.69 | 30.88 |
| 5. | Hydroxypropyl Methyl Cellulose | 25.19 | 26.52 | — |
| | Phthalate | | | |
| 6. | Fumaric Acid | 5.04 | — | — |
| 7. | Hydroxypropyl Methylcellulose | 10.08 | 10.61 | 14.44 |
| 8. | Glyceryl Behenate | 15.11 | 15.91 | 21.66 |

*Formulation does not contain hydroxypropyl methylcellulose phthalate and fumaric acid and serves as a control.

### Procedure:

### Formulation A:

Guanfacine hydrochloride was dry mixed with lactose, microcrystalline cellulose, hydroxypropyl methylcellulose phthalate to form a blend. The blend thus obtained was granulated with polyvinyl pyrrolidone. The granules thus obtained were dried and suitably sized by milling. The dried granules were mixed with fumaric acid and hydroxypropyl methyl cellulose to form the final blend. The final blend thus obtained was lubricated with glyceryl behenate and compressed into tablets using suitable tooling.

### Formulation B:

Guanfacine hydrochloride was dry mixed with lactose, microcrystalline cellulose, hydroxypropyl methylcellulose phthalate and hydroxypropyl methylcellulose to form a blend. The blend thus obtained was granulated with polyvinyl pyrrolidone. The granules thus obtained were dried and suitably sized by milling. The dried granules were lubricated with glyceryl behenate and compressed into tablets using suitable tooling. The compressed tablets thus obtained were coated with coating material as per the formula given above.

### Formulation C:

Guanfacine hydrochloride was dry mixed with lactose and microcrystalline cellulose and to form a blend. The blend thus obtained was granulated with polyvinyl pyrrolidone. The granules thus obtained were dried and suitably sized by milling. The dried granules were mixed with hydroxypropyl methylcellulose to form the final blend. The final blend thus obtained was lubricated with glyceryl behenate and compressed into tablets using suitable tooling.

The comparative dissolution data between formulations A, B and C in 0.1 N HCl (pH 1.2) and pH 6.8 buffer using paddle method (USP Apparatus II) employing 500 mL of dissolution media at a temperature of 37° C. and 50 rpm is given in Table 1 below:

TABLE 1

| % Drug Release for | | |
|---|---|---|
| Formulations A, B and C | | |
| in Acidic and Alkaline Media | | |
| A | B | C |

| Time (hr.) | 1* | 2^{#} | 1* | 2* | 1* | 2^{#} |
|---|---|---|---|---|---|---|
| 0.5 | 18 | 7 | 29 | 9 | 23 | 14 |
| 1.0 | 27 | 10 | 38 | 15 | 37 | 21 |
| 2.0 | 42 | 17 | 47 | 16 | 56 | 34 |
| 3.0 | 54 | 24 | 59 | 22 | 71 | 46 |
| 4.0 | 63 | 31 | 69 | 29 | 83 | 57 |
| 6.0 | 77 | 44 | 86 | 49 | 98 | 77 |
| 8.0 | 87 | 55 | 98 | 67 | 103 | 87 |
| 12.0 | 102 | 71 | 107 | 87 | 104 | 93 |

1* percent of guanfacine HCl dissolved using pH 0.1 dissolution medium
2^{#} percent of guanfacine HCl dissolved using pH 6.8 dissolution medium

The dissolution data of Table 1 shows that formulations A and B did not show any increase in the rate of release of guanfacine hydrochloride at a pH in excess of 5.5, as compared to the control formulation C.

### Example 9

| S.N. | Ingredients | Quantity (% w/w) |
|---|---|---|
| 1. | Guanfacine HCl | 0.5-3 |
| 2. | Carbopol | 12-34 |
| 3. | Microcrystalline Cellulose | 12-32 |
| 4. | Hydroxypropyl Methyl Cellulose | 8-20 |
| 5. | Ludipress^{#} | 12-32 |
| 6. | Povidone K-90 | 2-6 |
| 7. | Glyceryl Behenate | 8-16 |

^{#}Ludipress is composed of lactose (93.0% ± 2%), povidone K-30 (3.5% ± 0.5%) and crospovidone (3.5% ± 0.5%)

### Procedure:

Guanfacine hydrochloride is dry mixed with carbopol, microcrystalline cellulose, hydroxypropyl methylcellulose, ludipress and povidone K-90 to form a blend. The blend thus obtained is lubricated with glyceryl behenate. The lubricated blend is compressed into tablets using suitable tooling.

### Example 10

| S. N. | Ingredients | Quantity (% w/w) |
|---|---|---|
| 1. | Guanfacine HCl | 0.5-3 |
| 2. | Carbopol | 12-34 |
| 3. | Microcrystalline Cellulose | 12-32 |
| 4. | Hydroxypropyl Methyl Cellulose | 8-20 |
| 5. | Lactose | 12-32 |
| 6. | Povidone K-90 | 2-6 |
| 7. | Povidone K-30 | 1.6-4 |
| 8. | Glyceryl Behenate | 8-16 |

### Procedure:

Guanfacine hydrochloride is dry mixed with carbopol, microcrystalline cellulose, hydroxypropyl methylcellulose, lactose and povidone K-90 to form a blend. The blend thus obtained is granulated with povidone K-30. The granules thus obtained are dried and suitably sized by milling. The dried granules are lubricated with glyceryl behenate and compressed into tablets using suitable tooling.

We claim:

1. An extended release pharmaceutical tablet composition of guanfacine comprising:
a) a core containing guanfacine or a pharmaceutically acceptable salt thereof and one or more of pH-independent rate controlling polymer(s) and other pharmaceutically acceptable excipients; and
b) optionally a coating over the core of (a) wherein, the coating comprises one or more of pH-independent rate controlling polymer(s).

2. An extended release pharmaceutical tablet composition of guanfacine comprising:
a) an inert pellet;
b) a layer surrounding the inert pellet of (a), wherein the layer comprises guanfacine or a pharmaceutically acceptable salt thereof and other pharmaceutically acceptable excipients; and
c) a coating surrounding the layer of (b), wherein the coating comprises one or more of pH-independent rate controlling polymer(s) and other pharmaceutically acceptable excipients.

3. An extended release pharmaceutical tablet composition of guanfacine comprising guanfacine or a pharmaceutically acceptable salt thereof, one or more of sustained release wax, one or more of pH-independent rate controlling polymer(s) and other pharmaceutically acceptable excipients.

4. The extended release pharmaceutical tablet composition according to claims 1, wherein the core further comprises an acidic microenvironment pH modifier and/or enteric agent(s).

5. The extended release pharmaceutical tablet composition according to claim 1, wherein the core further comprises a gastro-soluble cationic polymethacrylate copolymer.

6. The extended release pharmaceutical tablet composition according to claim 1, wherein the core further comprises carboxyvinyl polymer.

7. The extended release pharmaceutical tablet composition according to claim 1, 2 or 3 wherein the pH-independent rate controlling polymer(s) comprises one or more of guar gum, acacia gum, tragacanth gum, xanthum gum, hydroxyethyl cellulose, hydroxypropyl cellulose, hydroxypropyl methylcellulose, carboxymethyl cellulose, sodium carboxymethyl cellulose, carbopol, polycarbofil, vinyl acetate copolymers, methacrylic acid copolymers, maleic anhydride-methyl vinyl ether copolymers, acrylates, ethylcellulose, methacrylates, acrylic acid copolymers, high molecular weight polyvinyl alcohols, stearyl alcohol, glyceryl palmitostearate, gleceryl monostearate, carnuba wax, beeswax, candelilla wax, microcrystalline wax, ozokerite wax, paraffin wax, glyceryl behenate and hydrogenated vegetable oil.

8. The extended release pharmaceutical tablet composition according to claim 4, wherein the acidic microenvironment pH modifier comprises one or more of hydrochloric acid, phosphoric acid, nitric acid, sulphuric acid, fumaric acid, citric acid, L-cysteine hydrochloride, glycine hydrochloride, ascorbic acid, butylated hydroxyanisol, butylated hydroxytoluene, salts of organic bases, salts of inorganic acids, salts of organic acids and acidic buffers.

9. The extended release pharmaceutical tablet composition according to claim 4, wherein the enteric agent(s) comprises one or more of hydroxypropyl methylcellulose phthalate, cellulose acetate phthalate, polyvinyl acetate phthalate.

10. The extended release pharmaceutical tablet composition according to claim 1, 2, or 3 wherein the other pharmaceutically acceptable excipients comprise one or more of binder(s), disintegrant(s), lubricant(s), diluent(s), glidants(s), surfactant(s), and solvent(s).
